(19) United States
(12) Patent Application Publication
(10) Pub. No.: US 2006/0235435 A1
(43) Pub. Date: Oct. 19, 2006

(54) Device for localization of stereotactic coordinates

(76) Inventors: Jens Christian Hedemann Soerensen, Egaa (DK); Graziano Cancian, Silkeborg (DK)

Correspondence Address: JACOBSON HOLMAN PLLC, 400 SEVENTH STREET N.W., SUITE 600, WASHINGTON, DC, 20004, US

(21) Appl. No.: 10/540,985

(22) PCT Filed: Dec. 18, 2003
(86) PCT No.: PCT/DK03/00894

§ 371 Date: Apr. 24, 2006

(30) Foreign Application Priority Data
Dec. 27, 2002 (DK) PA200202009

(51) Int. Cl. A61B 19/00

(52) U.S. Cl. 606/130

(57) ABSTRACT

The invention concerns a device for localization of stereotactic coordinates by available MRI, CT, PET and angiogram imaging equipment in relation to known art animal stereotactic frames comprised of a pair of bars held in spaced parallel relation to each other by a cross bar that may be fixed to an operating table or the like by means of a fixing device, which connects to the instrument. The instrument may be detached from the stereotactic frame to allow for the imaging procedures. The instrument is made of non-magnetic and radiolucent material allowing for the use of magnetic material and radio-opaque stereotactic frames in combination with available MRI, CT, PET, and angiogram imaging equipment.

The present invention relates to a fiducial marker instrument for identifying the spatial position of a region of interest in an individual's skull, which is to be subjected to stereotactic procedures. The position of the region of interest being determined for example by means of MRI, CT, PET, or X-ray equipment. The instrument comprises a box of nonmagnetic, radiolucent material with embedded fiducial markers attached to a non-yielding stereotactic frame. The steretoactic frame is arranged to be supported by the individuals skull and to support an indicating device and/or a treatment instrument, and fixing means, which are displaceably attached to the frame and which are arranged to engage the skull for operative procedures.

In surgical operations on the skull, as well as in non-invasive (bloodless) treatment of the brain, such as radiation treatment or heat treatment, the possibility of identifying with great accuracy the position of the area(s) of the patient's skull which is/are to be treated is highly important. To minimise the risk of unnecessary brain damage arising as a result of the treatment, the surgeon must be sure to hit, with reasonable accuracy, the region of interest, without having to make unnecessary passages through sensitive tissue.

The use of a scull fixed external fiducial marker system has been previously described.

The international patent application No. WO88/08282, which corresponds to U.S. Pat. No. 5,116,344, discloses a stereotactical instrument according to the first paragraph of the description. This instrument is also described by Leksell and Jernberg 1980, and in the brochure “Leksell Micro-stereotactic System”, from Elekta Instrument AB, to which reference is hereby made. It here appears that the spatial position of the area of interest, which is to be treated, is determined by means of angiogram, X-ray, PET, DSA, CT and/or MRI equipment, in relation to a box or cage construction with SUBSTITUTE SHEET (RULE 26) scales being used, for example, to obtain the coordinates of the treatment area in a patient's skull. For this purpose, said construction is attached to a non-yielding frame, which in turn is fixed to the skull with the aid of fixing means, which are displaceably attached to the frame and arranged to penetrate the skin so as to be attached to the bone of the skull. US 4,350,159, US 4,341,220 and US 4,475,550 disclose similar principles with detachable fiducial members associated with a stereotactical instrument having a non-yielding frame and skull fixing means. GB 2,370,778 discloses a non-invasive stereotactical instrument based on the Leksell Micro-stereotactic System. US 4,617,925 discloses a non-invasive stereotactical instrument, which is attached to a patient's skull and fixed by means of earplugs and a nose support. The patient may be fixed to an operating table or the like by means of a fixing device, which is connected to the instrument.

The known art applications all address treatment in human individuals. The human and primate scull has a spherical geometry as opposed to quadrupeds, which in general have sculls with a cylindrical geometry.

The present invention therefore employs a known art animal stereotactic frame comprising a pair of bars held in spaced parallel relation to each other by a cross bar that may be fixed to an operating table or the like by means of a fixing device, which is connected to the instrument. Such a frame is disclosed in US 3,841,148 and US 5,601,570.

By the invention, it is realised that by using the detachable box of nonmagnetic, radiolucent material with embedded fiducial markers, a number of advantages may be achieved. The fiducial marker box, hereafter referred to as the box, allows for fixation of sculls with a spherical geometry to known art animal stereotactic frames comprising a pair of bars held in spaced parallel relation to each other by a cross bar. An object of the present invention is to provide an external fiducial reference which may be fixed to the scull of individuals with cylindrical scull geometry. Furthermore, the invention is designed to be displaceably attached to a non-yielding stereotactic frame. The steretoactic frame is arranged to support an indicating device and/or a treatment instrument and fixing means, which are arranged to engage the skull for operative procedures.

A further object of the invention is to provide a stereotactical instrument, which does not impede the performance of the treatment and which allows the individual to remain mobile relative to the stereotactic frame.

Another object of the invention is to provide an external fiducial reference instrument, which is compatible with available MRI, CT, PET and angiogram equipment and the like and which is easy to displace and replace in known art animal stereotactic frames comprised of a pair of bars held in spaced parallel relation to each other by a cross bar.

The invention concerns a device for localisation of stereotactic coordinates by available MRI, CT, PET and angiogram imaging equipment in relation to known animal stereotactic frames comprised of a pair of bars held in spaced parallel relation to each other by a cross bar which may be fixed to an operating table or the like by means of a fixing device, which is connected to the instrument. The instrument may be detached from the stereotactic frame to allow for the imaging procedures. The instrument is made of non-magnetic and radiolucent material allowing for the use of magnetic material and radio-opague stereotactic frames in combination with available MRI, CT, PET and angiogram imaging equipment.

In the following, the invention is described with reference to the drawings, in which:

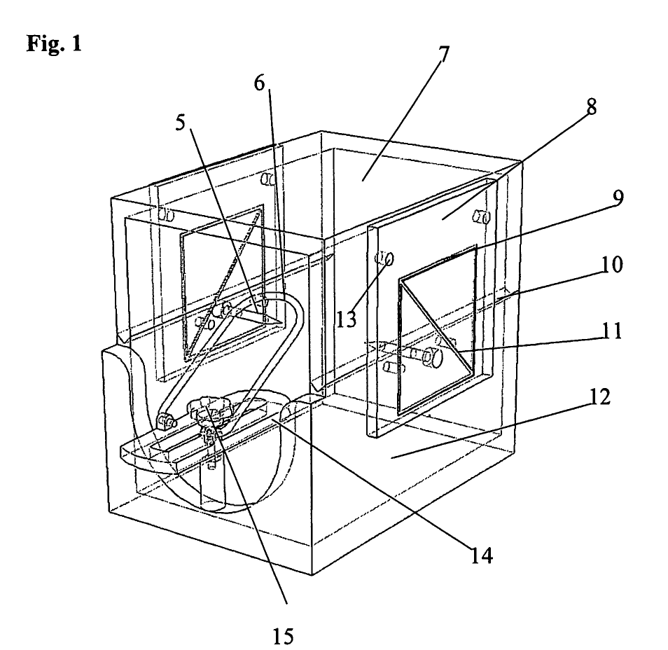

FIG. 1 is a perspective view of a device according to a preferred embodiment of the invention, and

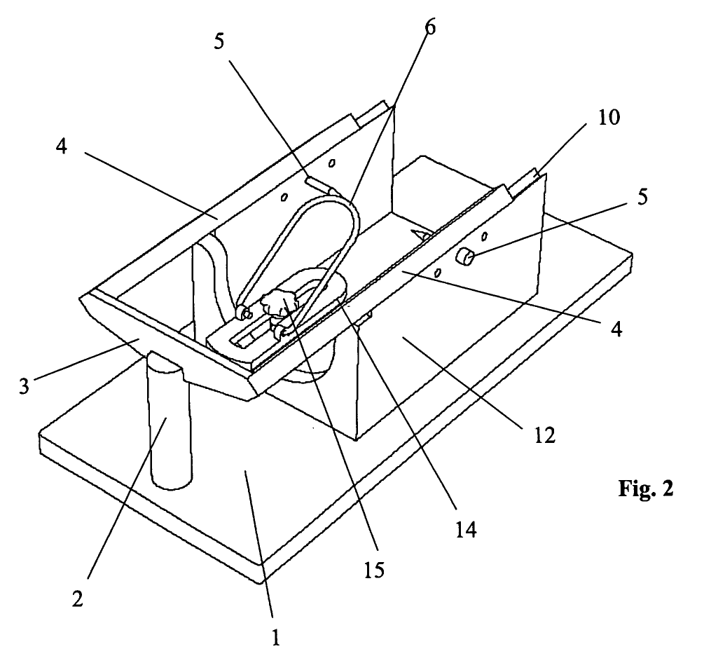

FIG. 2 is a perspective view of the device attached to a stereotactic frame.

FIG. 1 is a three-dimensional perspective view of the external fiducial reference instrument, which is made of a transparent and radiolucent material. The instrument consists of a top part 7 and bottom part 12. The two parts fit together with a ridge and groove 10 in the sides of the top part 7 and bottom part 12. Reference sign 5 show one embodiment of scull fixation pins for attachment of the external fiducial reference instrument to the scull of the individual in question, said scull fixation pins projecting inwardly into the interior space of the instrument. Reference sign 6 show one embodiment of a nose fixation clamp. The hard palate of the individual fits over the plate 14 and the clamp 6 is lowered over the nose ridge. The screw 15 may fix the nose and hard palate of the individual to the plate 14. The top part 7 may have an attachable side plate 8 made of a transparent and radiolucent material, according to the preferred embodiment of the invention. The side plate 8 comprises fiducial reference lines 9 and 11 so as to allow identification of the area of interest in available MRI, CT, PET and angiogram equipment.

FIG. 2 is a three-dimensional perspective view of the external fiducial reference instrument attached to the stereotactic frame. The known art animal stereotactic frame comprised a pair of bars 4 held in spaced parallel relation to each other by a cross bar 3 which could be fixed to an operating table 1 or the like by means of a fixing device 2, which was connected to the instrument as shown in US 3,841,148. The bottom part 12, with the top part 7 detached, will fit like a drawer under the parallel bars 4 of the stereotactic frame. The individual with the bottom part 12 attached to the scull may accordingly be transferred from the stereotactic frame to available MRI, CT, PET and angiogram equipment and back. When the individual is placed in available MRI, CT, PET and angiogram equipment, the top part 7 may be replaced on the bottom part 12 and kept in place by the ridge and grove 10. A locking mechanism may be applied to keep the top part 7 and bottom part 12 fixed to each other in one embodiment of the invention.

1. A device for localisation of stereotactic coordinates for identifying a spatial position of a region of interest in an individual's skull for the performance of stereotactic procedures, said device comprising a box of non-magnetic, radiolucent material with fiducial markers and said device being attachable to a stereotactic frame.

2. A device according to claim 1, wherein said fiducial markers includes scull fixation pins projecting inwardly into the interior space of the box.

3. A device according to claim 1 (or 2], wherein said box including at least one attachable side plate means made of translucent or transparent and radiolucent material.

4. A device according to claim 3, wherein said at least one side plate means further including fiducial reference lines allowing for identification of an area of interest.

5. A device according to any of tho preeding claims claim 1, wherein said device comprises a nose fixation clamp.

6. A device according to any of tho preceding ClaiLL claim 1, wherein said device is adapted to fit like a drawer under the stereotactic frame, when being attached thereto.

7. A device according to any of tho preceding claims claim 1, wherein said box comprises a top part and a bottom part fitting together with a ridge and groove in the sides of the top part and the bottom part.

8. A device according to claim 7, wherein said box is provided with a locking mechanism to allow for the top and bottom parts being fixed to each other.
